(19) United States
(12) Patent Application Publication
(10) Pub. No.: US 2026/0234679 A1
(43) Pub. Date: Aug. 13, 2026

(54) BIOSYNTHESIS OF 3-INDOXYL PHOSPHATE FROM 3-HYDROXYINDOLE USING KINASES AND INDIGO DYEING METHOD USING THE SAME

(71) Applicant: SEOUL NATIONAL UNIVERSITY R&DB FOUNDATION, Seoul (KR)

(72) Inventors: Byung Gee Kim, Seoul (KR); Sang Woo Seo, Seoul (KR); Jeongchan Lee, Seoul (KR)

(73) Assignee: SEOUL NATIONAL UNIVERSITY R&DB FOUNDATION, Seoul (KR)

(21) Appl. No.: 19/152,389

(22) PCT Filed: Jan. 31, 2023
(86) PCT No.: PCT/KR2023/001411

§ 371 Date: Apr. 10, 2026

(30) Foreign Application Priority Data
Jan. 30, 2023 (KR) 10-2023-0011930

(51) Int. Cl. C12P 17/10; C09B 7/02; C12P 17/16; D06P 1/22

(52) U.S. Cl. CPC C12P 17/10; C09B 7/02; C12P 17/165; D06P 1/228

(57) ABSTRACT

The present invention proposes an efficient production and dyeing method of an eco-friendly indigo precursor using indoxyl phosphate having a phosphoryl group as a functional group, thereby proposing a method for overcoming the problems of conventional indican and indoxyl acetates. To this end, the present invention found and characterized a novel kinase capable of producing 3-indoxyl phosphate from 3-hydroxyindole for the biosynthesis of 3-indoxyl phosphate, and also confirmed that when indigo denim fabric is dyed using indoxyl phosphate and alkaline phosphatase, a dyeing efficiency almost similar to that of chemical indigo dye is exhibited.

### TECHNICAL FIELD

The present invention relates to a method for efficient preparation of 3-indoxyl phosphate from 3-hydroxyindole using kinases and dyeing fabric using the same.

### BACKGROUND TECHNOLOGY

Due to the vivid blue color of indigo and the stability of the dye, approximately 50,000 tons of indigo are produced annually and used for dyeing fabrics. Although other blue dyes have been synthesized and developed, none have yet replaced indigo as a denim dye.

However, the high demand for such indigo dye presents two issues associated with environmental concerns. First, industrial-scale indigo synthesis is carried out through an environmentally hazardous process that involves the use of aniline, hydrogen cyanide, and formaldehyde with sodium amide under strongly basic conditions. The chemicals used in the synthesis are derived from environmentally harmful petroleum-based or toxic substances, and this produces a large amount of hazardous wastewater.

Second, in the indigo dyeing process, indigo is solubilized into its reduced form, leuco-indigo, to enable the dye to penetrate the cotton fabric; at this stage, a strong reducing agent such as sodium dithionite (Na₂S₂O₄) is required. The process using Na₂S₂O₄ is relatively inexpensive and has a short reduction time; however, the sulfites and sulfates produced after the reduction process must be removed through additional wastewater treatment, resulting in the production of large volumes of wastewater. It is known that about 12,000 L of wastewater is generated per pair of jeans, and considering the annual consumption volumes of indigo (50,000 tons/year) and jeans (5 billion pairs/year), the current indigo dyeing process generates a substantial amount of wastewater, causing environmental pollution.

To solve these environmental problems, there have been attempts to produce indigo dye using microorganisms. For example, by using microorganisms such as Esherichia coli or Corynebacterium glutamicum, indigo can be prepared from naturally occurring tryptophan and glucose without toxic chemicals.

However, indigo produced through the microbial process still requires a large amount of Na₂S₂O₄ reducing agent to solubilize the dye during the dyeing process. In addition, due to its strong hydrophobicity, the produced indigo inserts into cell membranes and accumulates inside the cells, necessitating further purification of the produced indigo. As a result, indigo produced through microorganisms has not been put into practical use as it still causes a large amount of wastewater and environmental pollution.

In order to overcome the issue of using a large amount of strong reducing agents in the indigo dyeing process, methods have recently been proposed that use indigo derivatives designed for good water solubility. For example, a method of dyeing denim fabrics using indigo carmine (5,5′-indigodisulfonic acid), an aromatic sulfonated indigo, is known. Through this method, the solubility of the indigo dye is improved, thereby reducing the amount of strong reducing agent used, which reduces the amount of wastewater. However, since the dye is still produced using toxic chemicals such as aniline, environmental concerns remain.

In order to solve the aforementioned two problems related to the indigo production and dyeing system, methods have been proposed that produce an indigo precursor from tryptophan or glucose in E. coli and use a protecting group to stabilize the unstable precursor (Non-Patent Literatures 1 and 2). For example, by using a glycosyl or acetyl group as a protecting group, indoxyl, an unstable precursor of indigo, can be immediately protected after being generated from tryptophan. Thereafter, the protected indoxyl, that is, indican or indoxyl acetate, can freely penetrate the E. coli cell membrane. Therefore, after culturing the cells, only the supernatant of the medium containing indican or indoxyl acetate can be obtained and used as a dye. During dyeing, the cotton fabric is immersed in the culture supernatant containing the protected indigo precursor. The protecting group is then removed to generate indoxyl, which is oxidized within the fabric to form indigo, thereby achieving the dyeing effect. This strategy not only enables an environmentally friendly method for producing indigo based on natural resources such as tryptophan and glucose, but also drastically reduces wastewater by eliminating the use of a strong reducing agent for dissolving indigo.

However, despite such advantages, there are some limitations to using indican or indoxyl acetate for indigo production and dyeing. In the case of indican, UDP-glucosyltransferase derived from the Polygonum tinctorium plant is used to prepare indican from indoxyl. UDP-glucose is used for glycosylation, and it is synthesized inside the cell by consuming one glucose molecule and two ATP molecules. Accordingly, a large amount of feedstock ultimately ends up as wastewater instead of being converted into the target molecule, indigo, making the production process of indican somewhat inefficient. In addition, since β-glucosidase, which decomposes the glycosyl portion of indican, does not function at the optimal pH of about 11 during indigo dyeing, the dyeing quality of fabrics using indican is relatively inferior to that using chemically synthesized indigo.

Indoxyl acetate was proposed as an alternative for indigo production and dyeing to overcome such disadvantages of using indican. Indoxyl acetate is prepared from indoxyl using the activity of chloramphenicol acetyltransferase of Clostridium scindens. In the process of preparing indoxyl acetate, acetyl-CoA is used, which consumes one glucose molecule inside the cell to produce two acetyl-CoAs. Therefore, the preparation of indoxyl acetate in E. coli can be carried out more efficiently than the preparation of indican using UDP-glucose. In addition, in the dyeing process, the acetyl group can be easily removed using a base without the need for a separate enzyme, enabling dyeing at a high pH (approximately 11) and thereby ensuring good quality indigo dyeing. However, the low solubility and low stability of indoxyl acetate in water present problems in applying indoxyl acetate in actual textile dyeing processes.

### PRIOR ART DOCUMENTS

### Non-Patent Literatures

[Non-Patent Literature 1] Hsu, T. M., Welner, D. H., Russ, Z. N., Cervantes B., Prathuri, R. L., Adams, P. D. & Dueber, J. E. (2018) Employing a biochemical protecting group for a sustainable indigo dyeing strategy. Nat. Chem. Biol., 14, 256-261.

[Non-Patent Literature 2] Latimer, L. N., Russ, Z. N., Lucas, J. & Dueber, J. E. (2010) Exploration of Acetylation as a Base-Labile Protecting Group in Escherichia coli for an Indigo Precursor. ACS Synth. Biol., 9(10), 2775-2783.

### SUMMARY OF INVENTION

### Technical Problem

The objective of the present invention is to provide an environmentally friendly and high-efficiency fabric dyeing method, and an efficient method for preparing 3-indoxyl phosphate for use therein.

### Solution to Problem

The present inventors have confirmed that the use of 3-indoxyl phosphate may serve as an environmentally friendly and efficient alternative to the conventional indigo production and dyeing processes, in order to overcome all the aforementioned problems.

Since 3-indoxyl phosphate can be prepared from indoxyl by a kinase using ATP, it has the advantage of being capable of producing indigo more efficiently than indican using UDP-glucose, and being more readily soluble in water and being stable without easily decomposing, compared to indoxyl acetate.

In addition, alkaline phosphatase is used to decompose 3-indoxyl phosphate and produce indoxyl during the dyeing process, and since the optimal pH for the operation of this phosphatase is 11, it can operate at the optimal pH for indigo dyeing, thereby ensuring high indigo dyeing efficiency.

Accordingly, in the present invention, kinases exhibiting activity toward low molecular-weight compounds such as indoxyl were screened to identify a kinase effective for the preparation of 3-indoxyl phosphate, and by applying this kinase to the production and dyeing process of indigo, the problems associated with conventional indigo production and dyeing processes were addressed.

Specifically, the present invention provides the following:
(1) A method for preparing 3-indoxyl phosphate, comprising reacting a kinase or a recombinant microbial strain expressing a kinase with 3-hydroxyindole.
(2) The method according to (1), wherein the kinase is 4-hydroxytryptamine kinase.
(3) The method according to (2), wherein the 4-hydroxytryptamine kinase is derived from Psilocybe cubensis.
(4) The method according to (1), wherein the reaction is carried out in the presence of a reducing agent and ATP.
(5) The method according to (4), wherein the reducing agent is beta-mercaptoethanol, glutathione reduced, or DL-dithiothreitol.
(6) The method according to (4), wherein the concentration of ATP is in the range of 1 to 20 mM.
(7) The method according to (1), wherein the reaction is carried out in a pH range of 7 to 9.
(8) The method according to (1), wherein the reaction is carried out at a temperature of 20 to 40° C.
(9) The method according to (1), wherein the recombinant microbial strain is Escherichia coli.
(10) A method for dyeing fabric, comprising the following:
a) reacting a kinase or a recombinant microbial strain expressing a kinase with 3-hydroxyindole to produce 3-indoxyl phosphate;
b) adding an alkaline phosphatase or a recombinant microbial strain expressing an alkaline phosphatase to the supernatant of the reaction mixture obtained in step a) to produce indigo; and
c) dyeing a fabric by immersing the fabric in the supernatant of the reaction mixture obtained in step b).
(11) The method according to (10), wherein the kinase is 4-hydroxytryptamine kinase.
(12) The method according to (11), wherein the 4-hydroxytryptamine kinase is derived from Psilocybe cubensis.
(13) The method according to (10), wherein step a) is carried out in the presence of a reducing agent and ATP.
(14) The method according to (13), wherein the reducing agent is beta-mercaptoethanol, glutathione reduced, or DL-dithiothreitol.
(15) The method according to (13), wherein the concentration of ATP is in the range of 1 to 20 mM.
(16) The method according to (10), wherein step a) is carried out in a pH range of 7 to 9.
(17) The method according to (10), wherein step b) is carried out in a pH range of 7 to 11.
(18) The method according to (10), wherein step a) and step b) are carried out simultaneously.
(19) The method according to (10), wherein the reactions in steps a) to c) are all carried out at a temperature of 20 to 40° C.
(20) The method according to (10), wherein the alkaline phosphatase is derived from Escherichia coli.
(21) The method according to (10), wherein the recombinant microbial strain is Escherichia coli.
(22) The method according to (10), wherein the fabric is denim.

### Effect of the Invention

The present invention proposes an alternative strategy to the conventional industrial process by using a phosphate group as a protecting group of indoxyl, an indigo precursor (FIG. 1). First, as a result of screening 13 types of bacterial-derived kinases capable of biosynthesizing 3-indoxyl phosphate from 3-hydroxyindole, it was confirmed that 4-hydroxytryptamine kinase from Psilocybe cubensis exhibited the highest activity. In addition, as a result of using the biosynthesized 3-indoxyl phosphate as a denim dye, 3-indoxyl phosphate showed a dyeing quality comparable to that of chemically synthesized indigo.

When 3-indoxyl phosphate is prepared from tryptophan or sugar using a recombinant microorganism expressing such a kinase, indigo dye can be produced from an environmentally friendly material using microorganisms, and since a strong reducing agent is not used in the dyeing process, indigo dyeing is possible in an environmentally friendly way that does not generate wastewater.

In summary, the present invention is the first invention on a method for dyeing fabric with indigo dye using 3-indoxyl phosphate, and the first invention on a method for biosynthesizing 3-indoxyl phosphate using an enzyme. Through the present invention, it is expected that both the conventional indigo production and dyeing processes may be replaced in the future with environmentally friendly alternatives.

### BRIEF DESCRIPTION OF DRAWINGS

FIG. 1 shows an outline of a process for producing indigo dye required for dyeing denim by biosynthesizing 3-indoxyl phosphate from 3-hydroxyindole using a kinase and then reacting the same with an alkaline phosphatase.

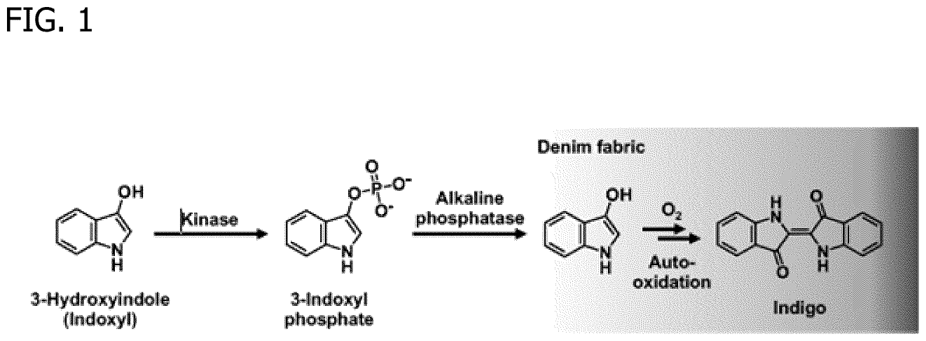

FIG. 2 shows the names, origins, and known substrates of kinase candidates for the biosynthesis of 3-indoxyl phosphate.

FIG. 3 is a schematic diagram for the reaction in biosynthesis of 3-indoxyl phosphate (3-IP) from indican.

FIG. 4 is a graph showing the amount of 3-indoxyl phosphate produced from 3-hydroxyindole by the 13 kinase candidates.

FIG. 5 is a graph showing the amount of 3-indoxyl phosphate produced by the hour by 4-hydroxytryptamine kinase (PsiK) derived from Psilocybe cubensis.

FIG. 6 is a graph showing the difference in the amount of 3-indoxyl phosphate produced according to changes in the reaction pH, ATP concentration, and type of reducing agent of PsiK kinase.

FIG. 7 is a liquid chromatography-mass spectrometry (LC-MS) analysis result of 3-indoxyl phosphate produced by PsiK kinase.

FIG. 8 shows the results confirming the production of indigo from 3-indoxyl phosphate using 3-indoxyl phosphate and alkaline phosphatase according to changes in time and the concentration of 3-indoxyl phosphate.

FIG. 9 is a photograph showing the process of dyeing denim fabric using an aqueous solution of 3-indoxyl phosphate and an E. coli cell lysate expressing an alkaline phosphatase.

FIG. 10 is a table showing the color values of indigo dyed fabric using 3-indoxyl phosphate and fabric dyed using an indigo reference standard before and after washing.

### DESCRIPTION OF EMBODIMENTS

Hereinafter, the present invention will be described in more detail. Various modifications may be made to the Examples described below. The Examples described below are not intended to limit the embodiments, and should be understood to include all modifications and alternatives thereof.

Unless otherwise defined, the terms used in the present invention are the same as those commonly used in the relevant technical field. General terms should be interpreted to have the meanings consistent with their context in the relevant art and should not be construed in an idealized or unduly broad manner.

The terms used in the Examples are not intended to limit a specific situation, but are intended to describe the examples. “3-IP” used in the Examples of the present invention is an abbreviation for 3-indoxyl phosphate, “PhoA” is an abbreviation for alkaline phosphatase, and “LAA,” “BME,” “GSH,” and “DTT” are reducing agents used in the reaction and abbreviations for L-ascorbic acid, beta-mercaptoethanol, glutathione reduced, and DL-dithiothreitol, respectively.

One embodiment of the present invention relates to a method for preparing 3-indoxyl phosphate, comprising reacting a kinase or a recombinant microbial strain expressing a kinase with 3-hydroxyindole.

The reaction may be carried out by a purified kinase, or by a recombinant microbial strain expressing a kinase, or a cell lysate obtained therefrom.

The kinase of the present invention refers to an enzyme having activity to introduce a phosphate group into 3-hydroxyindole. For example, the kinase of the present invention is 4-hydroxytryptamine kinase or methylthioribose kinase, preferably, 4-hydroxytryptamine kinase derived from Psilocybe cubensis, methylthioribose kinase derived from Bacillus subtilis, methylthioribose kinase derived from Bacillus licheniformis, methylthioribose kinase derived from Bacillus cereus, or methylthioribose kinase derived from Bacillus thuringiensis, and more preferably, 4-hydroxytryptamine kinase derived from Psilocybe cubensis, but is not limited thereto.

The aforementioned reaction can be carried out in the presence of a reducing agent and ATP. The reducing agent may be, for example, beta-mercaptoethanol, glutathione reduced, or DL-dithiothreitol, but is not limited thereto. ATP can be used at a concentration suitable for the reaction catalyzed by a kinase. For example, ATP can be used at a concentration in the range of 1 to 20 mM.

The reaction can be performed under pH and temperature conditions suitable for the activity of the kinase. For example, the kinase reaction can be carried out in a buffer solution having a pH in the range of 7 to 9, and at a temperature of 20 to 40° C.

The microbial strain of the present invention includes all microbial strains used for the expression of recombinant proteins in the relevant field, and is preferably E. coli, but is not limited thereto.

Another embodiment of the present invention relates to a method for dyeing fabric, comprising the following:
a) reacting a kinase or a recombinant microbial strain expressing a kinase with 3-hydroxyindole to produce 3-indoxyl phosphate;
b) adding an alkaline phosphatase or a recombinant microbial strain expressing an alkaline phosphatase to the supernatant of the reaction mixture obtained in step a) to produce indigo; and
c) dyeing a fabric by immersing the fabric in the supernatant of the reaction mixture obtained in step b).

The reaction in step a) may be carried out by a purified kinase, or can be carried out by a recombinant microbial strain expressing a kinase, or a cell lysate obtained therefrom.

The reaction in step a) may be carried out in the presence of a reducing agent and ATP. The reducing agent may be, for example, beta-mercaptoethanol, glutathione reduced, or DL-dithiothreitol, but is not limited thereto. ATP may be used at a concentration suitable for the reaction catalyzed by a kinase. For example, ATP may be used at a concentration in the range of 1 to 20 mM.

The reaction in step a) can be carried out under pH and temperature conditions suitable for the activity of the kinase. For example, the kinase reaction can be carried out in a buffer solution in the range of pH 7 to 9, and at a temperature of 20 to 40° C.

The reaction in step b) can be carried out by a purified alkaline phosphatase, or by a recombinant microbial strain expressing an alkaline phosphatase, or a cell lysate obtained therefrom.

The alkaline phosphatase of the present invention is an enzyme having activity to hydrolyze a monoester of phosphoric acid to produce inorganic phosphate and alcohol, and refers to an enzyme that exhibits optimal activity under alkaline conditions. For example, the alkaline phosphatase of the present invention may be an alkaline phosphatase derived from E. coli, but is not limited thereto.

The reaction in step b) can be carried out under pH and temperature conditions suitable for the activity of the alkaline phosphatase. For example, the alkaline phosphatase reaction may be carried out in a buffer solution in the pH range of 7 to 11, preferably in a buffer solution in the pH range of 8 to 10, and at a temperature of 20 to 40° C.

The reaction in step b) may be carried out after the reaction in step a) is finished, or may be performed simultaneously with step a) in one reaction vessel.

Step c) is a step of dyeing a fabric with the indigo dye obtained in step b), and is carried out by separating a supernatant from the reaction mixture obtained in step b) by a method such as centrifugation, and then immersing the fabric in the supernatant.

Step c) may be carried out under pH and temperature conditions suitable for dyeing fabric. For example, step c) may be carried out in a pH range of 7 to 11, preferably, in a pH range of 10 to 11, and may also be carried out at a temperature of 20 to 40° C. In addition, step c) may be carried out for a sufficient period required for dyeing the fabric.

In the dyeing method according to the present invention, the fabric used as the dyeing material is silk, wool, acrylic, nylon, cotton, or denim, and preferably denim.

The present invention will be described in more detail through the following examples. Various changes may be made to the examples described below. The examples described below are not intended to limit the embodiments, and should be understood to include all modifications and substitutions thereof. The terms used in the examples are not intended to limit a specific situation, but are intended to describe the examples.

### EXAMPLES

### [Example 1] Screening and Characterization of Kinases for Biosynthesis of 3-indoxyl Phosphate

In order to biosynthesize 3-IP from 3-hydroxyindole, a kinase reactive to 3-hydroxyindole must be found. However, since 3-IP is not a substance that exists in nature, enzymes whose main activity is phosphorylation of 3-hydroxyindole to produce 3-IP are not known. Therefore, for such biosynthesis, an additional activity (promiscuous activity) of a known kinase must be utilized.

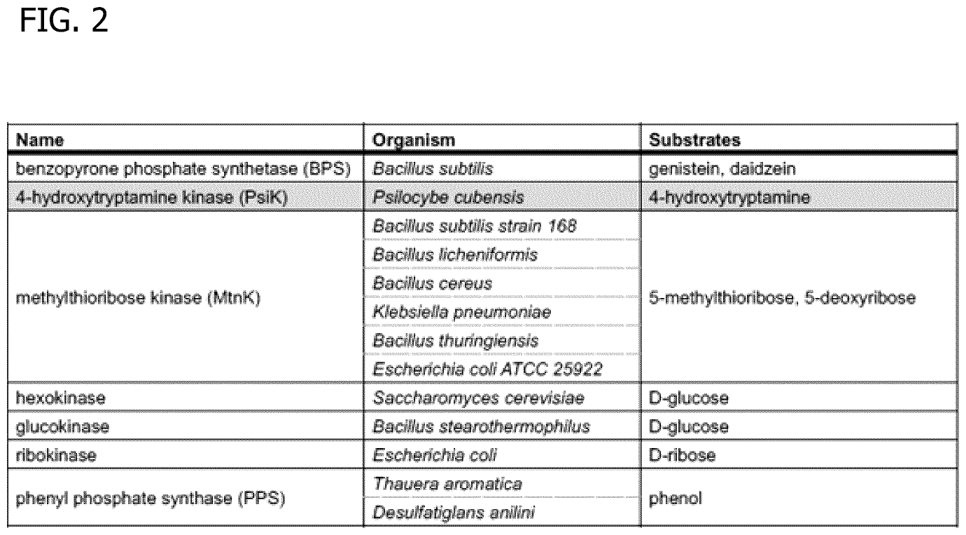

Considering the small molecular size of 3-hydroxyindole, 13 kinase candidates known to have small substrate sizes and to be reactive to the ring structure were selected among the known kinases (FIG. 2). The 13 kinase candidates are as follows:
Benzopyrone phosphate synthetase (BsBPS) from Bacillus subtilis
4-hydroxytryptamine kinase (PsiK) from Psilocybe cubensis
Methylthioribose kinase (BsMtnK) from Bacillus subtilis strain 168
Methylthioribose kinase (BlMtnK) from Bacillus licheniformis
Methylthioribose kinase (BcMtnK) from Bacillus cereus
Methylthioribose kinase (KpMtnK) from Klebsiella pneumoniae
Methylthioribose kinase (BtMtnK) from Bacillus thuringiensis
Methylthioribose kinase (EcMtnK) from Escherichia coli ATCC 25922
Hexokinase (ScHK) from Saccharomyces cerevisiae
Glucokinase (BsGK) from Bacillus stearothermophilus
Ribokinase (EcRK) from Escherichia coli
Phenyl phosphate synthase (TaPPS) from Thauera aromatica
Phenyl phosphate synthase (DaPPS) from Desulfatiglans anilini

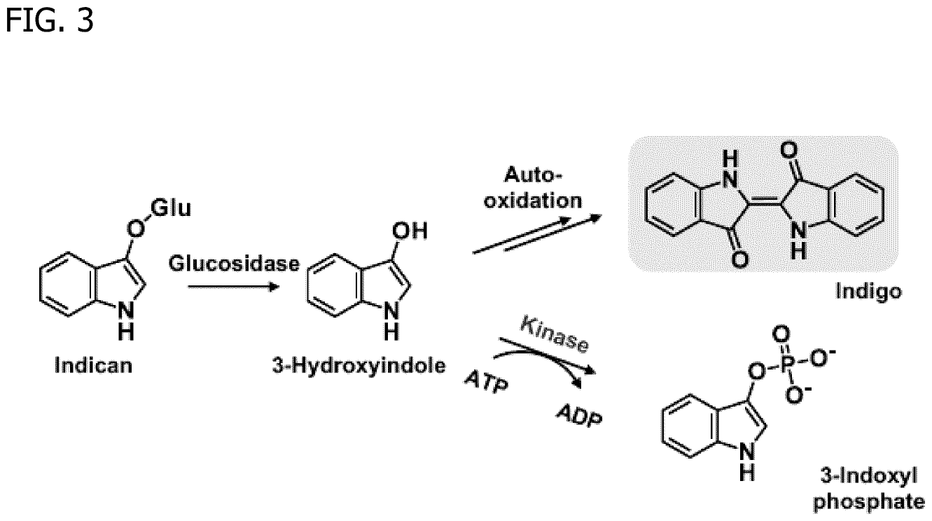

To screen enzymes that have reactivity toward 3-hydroxyindole from the above 13 kinases, 1 μM of each kinase was added to a sodium phosphate buffer solution (pH 8.0) containing 0.6 mM indican, 5 mM MgCl₂, 5 mM ATP, 10 mM DTT, and 1 μM β-glucosidase, and the reaction was carried at 37° C. for 3 hours, after which, the amount of 3-IP generated from 3-hydroxyindole was investigated. In this reaction, since the substrate 3-hydroxyindole is highly unstable and forms indigo through auto-oxidation, 3-hydroxyindole was produced from indican using β-glucosidase and then supplied to the phosphorylation enzyme (FIG. 3).

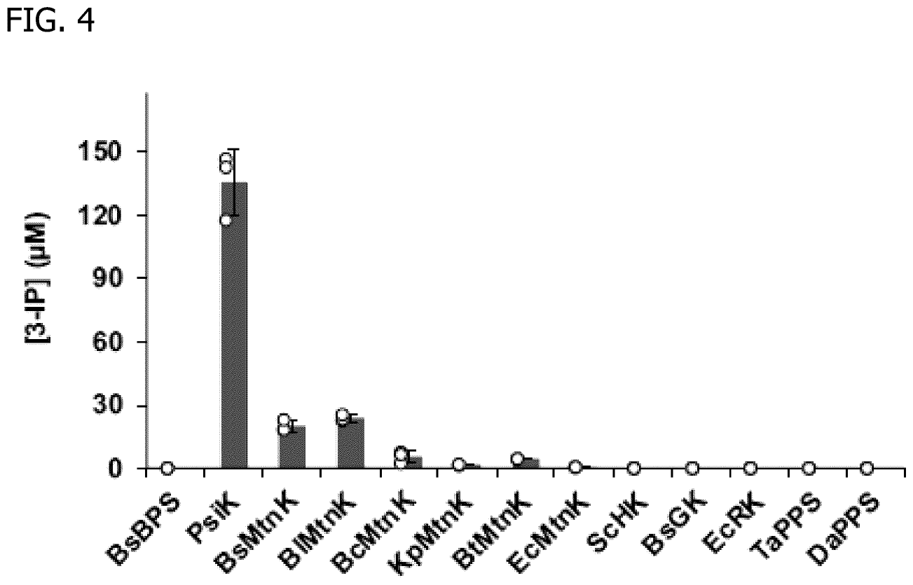

As a result of the above reaction, the preparation of 3-IP in PsiK, BsMtnK, BlMtnK, BcMtnK, KpMtnK, and BtMtnK was confirmed, and among them, the highest production of 3-IP was found to be in PsiK (FIG. 4, approximately 0.14 mM of 3-IP produced from 0.6 mM substrate, 23% conversion rate).

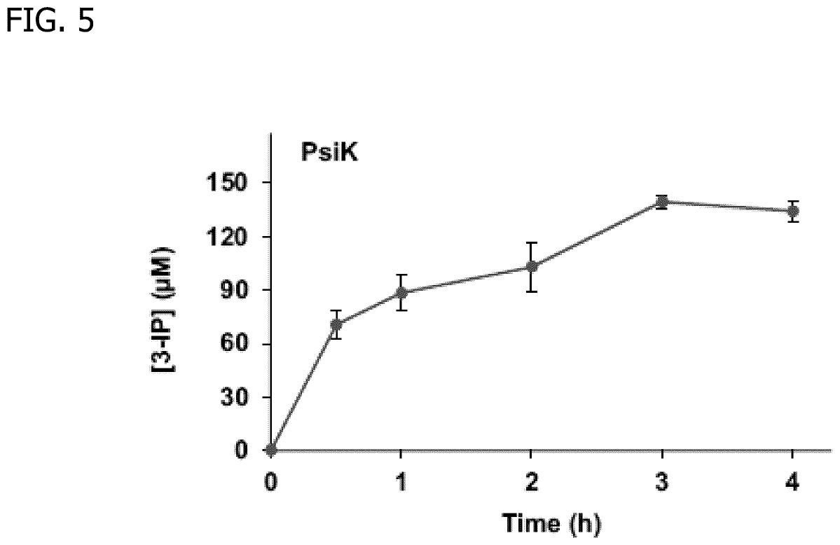

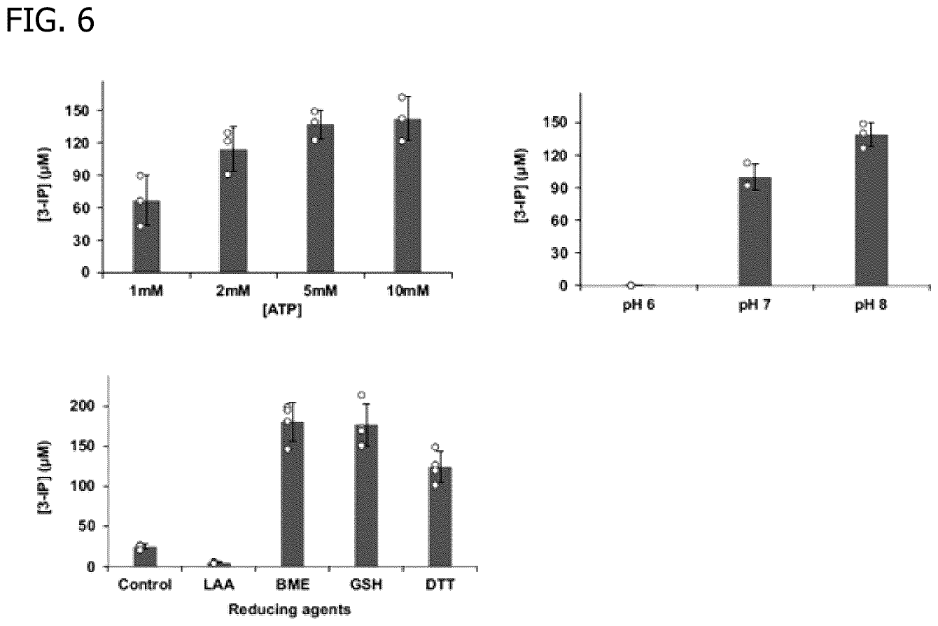

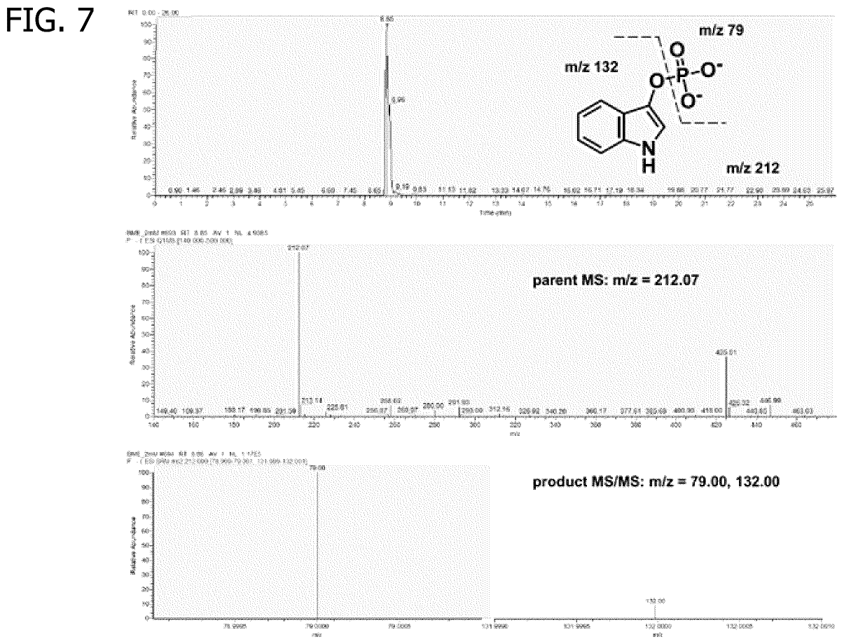

Subsequently, to characterize the PsiK-catalyzed reaction, the reaction time, ATP concentration, pH, and the type of reducing agent were varied, and the enzyme's reactivity under each condition was examined (FIG. 5 and FIG. 6). As a result, it was confirmed that the optimal conditions for 3-IP preparation were ATP at a concentration of 5 mM at pH 8.0 (sodium phosphate buffer solution) and using BME as a reducing agent, enabling the biosynthesis of 3-IP at a final concentration of 0.18 mM. The biosynthesized 3-IP was confirmed by LC/MS/MS analysis, where a parent ion at m/z 212 and product ions at m/z 79 and 132 of 3-IP were observed, thereby verifying that 3-IP was biosynthesized from 3-hydroxyindole by PsiK (FIG. 7).

### [Example 2] Method for Dyeing Denim Fabric Using 3-indoxyl Phosphate

The purpose was to confirm whether it was efficient to produce indigo dye from biosynthesized 3-IP and use it to dye denim. To this end, it was first confirmed whether 3-IP is hydrolyzed using PhoA, resulting in the formation of indigo.

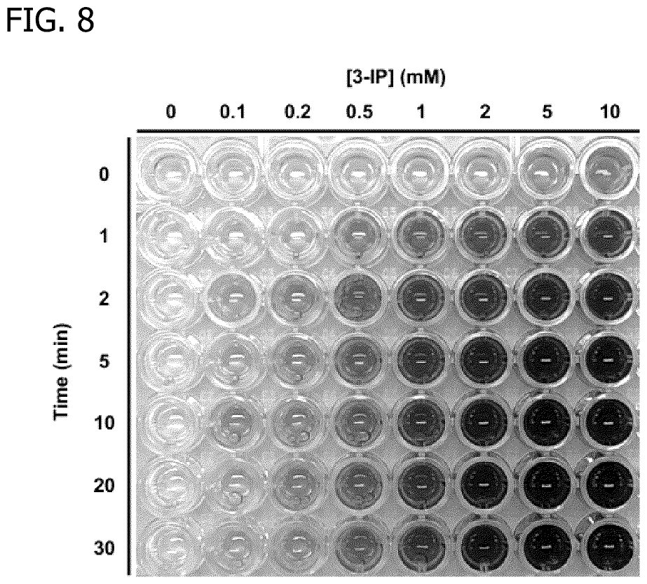

As a result, the hydrolysis of 3-IP by PhoA was carried out within 2 to 5 minutes (FIG. 8), and it was confirmed that this takes much less time than the indican hydrolysis process, which takes 30 minutes or more (Non-Patent Literature 2). Therefore, denim fabric can be dyed more efficiently in a shorter period of time when 3-IP is used than the conventional method for using indican in indigo dyeing.

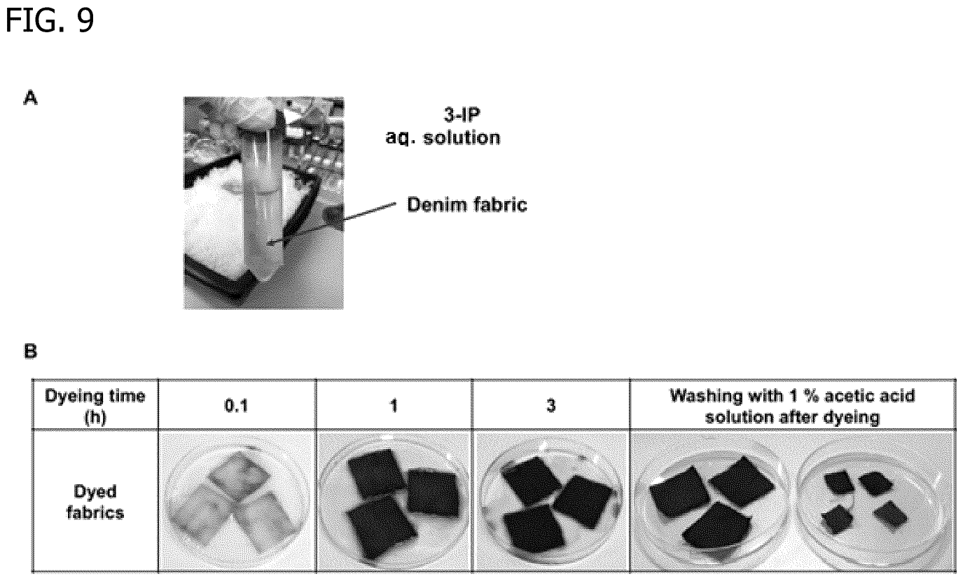

Next, in order to evaluate the characteristics of dyeing using 3-IP, denim fabric was dyed using a chemical dye, an indigo reference standard, as a control group (FIG. 9). Since two 3-IP molecules make one indigo molecule, dyeing was carried out using 3-IP so that it has the same molar number as the chemical indigo.

In order to compare the dyeing efficiency of fabrics dyed using 3-IP and fabrics dyed using an indigo reference standard, K/S (absorption coefficient K, scattering coefficient S) values were compared at the indigo absorption wavelength of 640-650 nm. In addition, K/S values before and after washing the fabrics were compared.

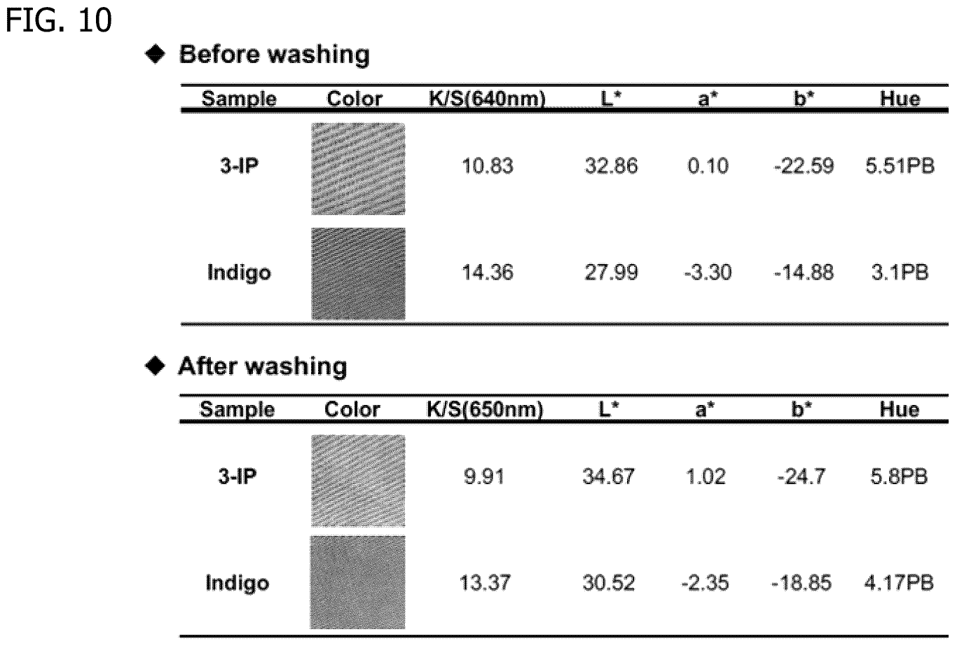

As a result, the K/S values of the fabrics dyed using 3-IP were slightly lower than those dyed using the indigo reference standard, while showing that they could have dyeing efficiency comparable to the chemical indigo dye. In addition, it was confirmed that there was almost no change in the K/S values before and after washing (FIG. 10).

Further, in order to quantitatively compare the brightness and color of the dyed fabrics, the brightness index (L*), red-green index (a*), and yellow-blue index (b*) were analyzed. It was confirmed that all values obtained using 3-IP and the indigo reference standard as dyes showed minimal differences and were nearly identical (FIG. 10). The above experimental results support the fact that color characteristics almost similar to when chemical indigo dyes are used can be exhibited through the method for dyeing based on use of 3-IP according to the present invention.

### INDUSTRIAL APPLICABILITY

Indigo dye is a dye found in plants, and it was discovered that derivatized indigo of various colors such as red, yellow, and various blues exist in nature through derivatization of indigo dyes. Therefore, if derivatization of 3-indoxyl phosphate is performed based on the present invention, indigo derivatives with various colors can be produced from microorganisms, and it is expected that various colors of textile dyes can be produced in an environmentally friendly manner.

In addition, since the blue color can give a refreshing feeling to cosmetic ingredients, and indigo is a natural product derived from plants that has been used for a long time and for which safety has been proven, there have been recent attempts to use indigo dyes as cosmetic dyes. In the present invention, unlike chemically synthesized indigo, indigo without any remaining toxic substances can be biosynthesized, thereby suggesting the potential for indigo dye to be used as a cosmetic ingredient without any residual harmful substances.

1. A method for preparing 3-indoxyl phosphate, comprising reacting a kinase or a recombinant microbial strain expressing a kinase with 3-hydroxyindole.

2. The method according to claim 1, wherein the kinase is 4-hydroxytryptamine kinase.

3. The method according to claim 2, wherein the 4-hydroxytryptamine kinase is derived from Psilocybe cubensis.

4. The method according to claim 1, wherein the reaction is carried out in the presence of a reducing agent and ATP.

5. The method according to claim 4, wherein the reducing agent is beta-mercaptoethanol, glutathione reduced, or DL-dithiothreitol.

6. The method according to claim 4, wherein the concentration of ATP is in the range of 1 to 20 mM.

7. The method according to claim 1, wherein the reaction is carried out in a pH range of 7 to 9.

8. The method according to claim 1, wherein the reaction is carried out at a temperature of 20 to 40° C.

9. The method according to claim 1, wherein the recombinant microbial strain is Escherichia coli.

10. A method for dyeing fabric, comprising the following steps:
a) reacting a kinase or a recombinant microbial strain expressing a kinase with 3-hydroxyindole to produce 3-indoxyl phosphate;
b) adding an alkaline phosphatase or a recombinant microbial strain expressing an alkaline phosphatase to the supernatant of the reaction mixture obtained in step a) to produce indigo; and
c) dyeing a fabric by immersing the fabric in the supernatant of the reaction mixture obtained in step b).

11. The method according to claim 10, wherein the kinase is 4-hydroxytryptamine kinase.

12. The method according to claim 11, wherein the 4-hydroxytryptamine kinase is derived from Psilocybe cubensis.

13. The method according to claim 10, wherein step a) is carried out in the presence of a reducing agent and ATP.

14. The method according to claim 13, wherein the reducing agent is beta-mercaptoethanol, glutathione reduced, or DL-dithiothreitol.

15. The method according to claim 13, wherein the concentration of ATP is in the range of 1 to 20 mM.

16. The method according to claim 10, wherein step a) is carried out in a pH range of 7 to 9.

17. The method according to claim 10, wherein step b) is carried out in a pH range of 7 to 11.

18. The method according to claim 10, wherein step a) and step b) are carried out simultaneously.

19. The method according to claim 10, wherein the reactions in steps a) to c) are all carried out at a temperature of 20 to 40° C.

20. The method according to claim 10, wherein the alkaline phosphatase is derived from Escherichia coli.

21. The method according to claim 10, wherein the recombinant microbial strain is Escherichia coli.

22. The method according to claim 10, wherein the fabric is denim.
